(19) United States
(12) Patent Application Publication
(10) Pub. No.: US 2023/0372305 A1
(43) Pub. Date: Nov. 23, 2023

(54) A METHOD FOR REDUCING ALLERGENS

(71) Applicant: AQDOT LIMITED, Cambridge (GB)

(72) Inventors: Roger James COULSTON, Cambridge (GB); Andrew Michael HOWE, Cambridge (GB); Jessica Alice LENDERYOU, Cambridge (GB); Nicolas Serge Jacky ESSELIN, Cambridge (GB)

(73) Assignee: AQDOT LIMITED, Cambridge (GB)

(21) Appl. No.: 18/030,336

(22) PCT Filed: Oct. 7, 2021
(86) PCT No.: PCT/GB2021/052602

§ 371 Date: Apr. 5, 2023

(30) Foreign Application Priority Data
Oct. 7, 2020 (GB) 2015926.5
Oct. 13, 2020 (GB) 2016220.2

(51) Int. Cl. A61K 31/4188; A61P 37/08

(52) U.S. Cl. CPC A61K 31/4188; A61P 37/08

(57) ABSTRACT

A non-therapeutic method for reducing or eliminating the allergic effect of an allergen. Also relates to a cucurbituril, a derivative thereof, a variant thereof or a mixture thereof for use in treating and/or preventing the allergic effect of an allergen.

The invention relates to a non-therapeutic method for reducing or eliminating the allergic effect of an allergen. The invention also relates to a cucurbituril, a derivative thereof, a variant thereof or a mixture thereof for use in treating and/or preventing the allergic effect of an allergen.

An allergen is a type of antigen that produces an abnormally vigorous immune response in which the immune system fights off a perceived threat that would otherwise be harmless to the body. Such a response is known as an allergic response. Antigens can be proteins, shorter oligopeptides and polysaccharides. Lipids and nucleic acids become antigens only when combined with proteins and polysaccharides. Whilst polysaccharides and lipids qualify as antigens, they are not immunogens because they do not elicit an immune response (activation of T-cells by antigen-presenting cells) on their own. Furthermore for a peptide to induce an immune response, it must be a large enough size since small peptides will also not elicit an immune response.

Allergies are treated in three different ways. The most common treatment is avoiding the allergen altogether. The second option is by using medication, such as an antihistamine, to treat the allergic symptoms, but not the allergy itself. The third treatment option is immunotherapy that affects the actual allergic condition by helping the body produce less Immunoglobulin E (IgE) and build long-term tolerance to the allergen. IgE are antibodies produced by the immune system which, in response to an allergen, travel to cells that release chemicals, causing an allergic response.

Common allergens include those which originate from the domestic cat, the domestic dog, and the dust mite.

There are eight cat protein allergens known as Fel d 1 to Fel d 8. Fel d 1 is the most prominent cat allergen, accounting for 96% of human cat allergies. All cats produce Fel d 1 including hypoallergenic cats. The main way these allergens are spread is through a cat's saliva or dander, which gets stuck on clothing. Fel d 1 is part of the secretoglobulin family, which are proteins found only in mammals, and is primarily secreted through the sebaceous glands. Fel d 4 and Fel d 7, which are lipocalins, are the most common cat allergens after Fel d 1 and are produced in the salivary glands and deposited on to cat dander during grooming.

Whilst there are seven recognised domestic dog protein allergens known as Can f 1 to Can f 7, Can f 1 and Can f 2, which are both lipocalins, are the two major allergens present in dog dander.

The protein allergens Der p 1 from the European house dust mite (Dermatophagoides pteronyssinus) and Der f 1 from the American house dust mite (Dermatophagoides farina) are major sources of house dust mite allergies in temperate climates.

Other protein allergens originate from tree and grass pollen such as Bet v 1, the major white birch (Betula verrucosa) pollen antigen, and Phl p 5, which originates from Timothy grass (Phleum pratense).

Barrow et al. (‘Cucurbituril-Based Molecular Recognition’, Chemical Reviews, 115, 22, 12320-12406 (2015)) reviews amino acid and short peptide recognition by selected cucurbiturils.

Non-protein allergens or sources of allergens include fragrances selected from the group consisting of amyl cinnamal (CAS No 122-40-7), benzyl alcohol (CAS No 100-51-6), cinnamyl alcohol (CAS No 104-54-1) citral (CAS No 5392-40-5), eugenol (CAS No 97-53-0), hydroxy-citronellal (CAS No 107-75-5), isoeugenol (CAS No 97-54-1), amylcin-namyl alcohol (CAS No 101-85-9), benzyl salicylate (CAS No 118-58-1), cinnamal (CAS No 104-55-2), coumarin (CAS No 91-64-5), geraniol (CAS No 106-24-1), hydroxy-methylpentylcyclohexenecarboxaldehyde (CAS No 31906-04-4), anisyl alcohol (CAS No 105-13-5), benzyl cinnamate (CAS No 103-41-3), farnesol (CAS No 4602-84-0), 2-(4-tert-butylbenzyl)propionaldehyde (CAS No 80-54-6), linalool (CAS No 78-70-6), benzyl benzoate (CAS No 120-51-4), citronellol (CAS No 106-22-9), hexyl cinnamaldehyde (CAS No 101-86-0), d-limonene (CAS No 5989-27-5), methylheptin carbonate (CAS No 111-12-6), 3-methyl-4-(2,6,6-tri-methyl-2-cyclohexen-1-yl)-3-buten-2-one (CAS No 127-51-5), Oak moss and treemoss extract (CAS No 90028-68-55), Treemoss extract (CAS No 90028-67-4) (Regulation (EC) No 1223/2009 of the European Parliament and of the Council of 30 Nov. 2009 on cosmetic products (Annex III)). Further allergen fragrances are Citrus sinensis peel oil (CAS No 97766-30-8/8028-48-6) (Limonene is an allergen), Lavendula officinalis oil (CAS No 8000-28-0/90063-37-9), Jasminum grandiflorum oil (CAS No 8022-96-6), Mentha piperita oil (CAS No 8006-90-4/84082-70-2), and rose flower oil (CAS No 90106-38-0/8007-01-0).

There is a long standing need for a means for counteracting or neutralising the allergen before the onset of an allergic reaction.

### SUMMARY OF THE INVENTION

In a first aspect of the invention, a non-therapeutic method for reducing or eliminating the allergic effect of an allergen is provided, the method comprising the step of bringing the allergen into physical contact with an effective amount of a cucurbituril, a derivative thereof, a variant thereof or a mixture thereof.

Reference to ‘allergic effect’ means such an effect in a human or a non-human animal such as a mammal. Examples of suitable non-human mammals are a domestic dog or a domestic cat.

In a second aspect of the invention, a cucurbituril, a derivative thereof, a variant thereof or a mixture thereof for use in treating or preventing the allergic effect of an allergen in a human or non-human animal (such as a mammal) is provided.

Whilst not being bound by theory, it is thought by the inventors that cucurbituril, being a barrel-shaped molecule with carbonyl groups at the entrances of the barrel, complexes with chemical groups on the allergen and disrupts its ability to provoke an allergic response.

### BRIEF DESCRIPTION OF THE INVENTION

The invention is illustrated with reference to:

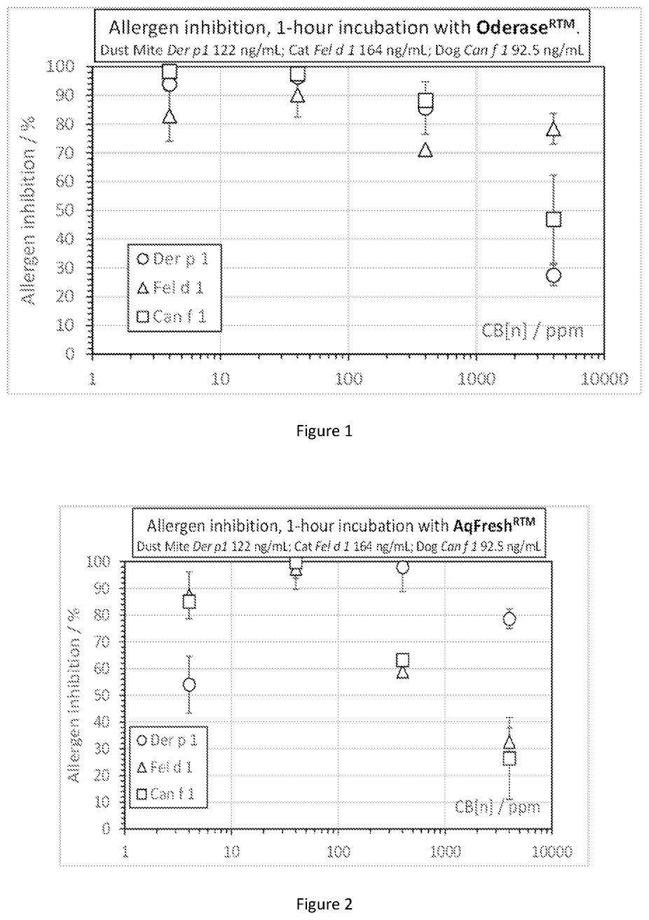

FIG. 1 which shows % allergen inhibition for various concentrations (ppm) of mixed cucurbiturils (CB[n]) in the air freshener marketed as Oderase® (Aqdot Limited, Cambridge, England);

FIG. 2 which shows % allergen inhibition for various concentrations (ppm) of mixed cucurbiturils (CB[n]) marketed as AqFresh® (Aqdot Limited, Cambridge, England);

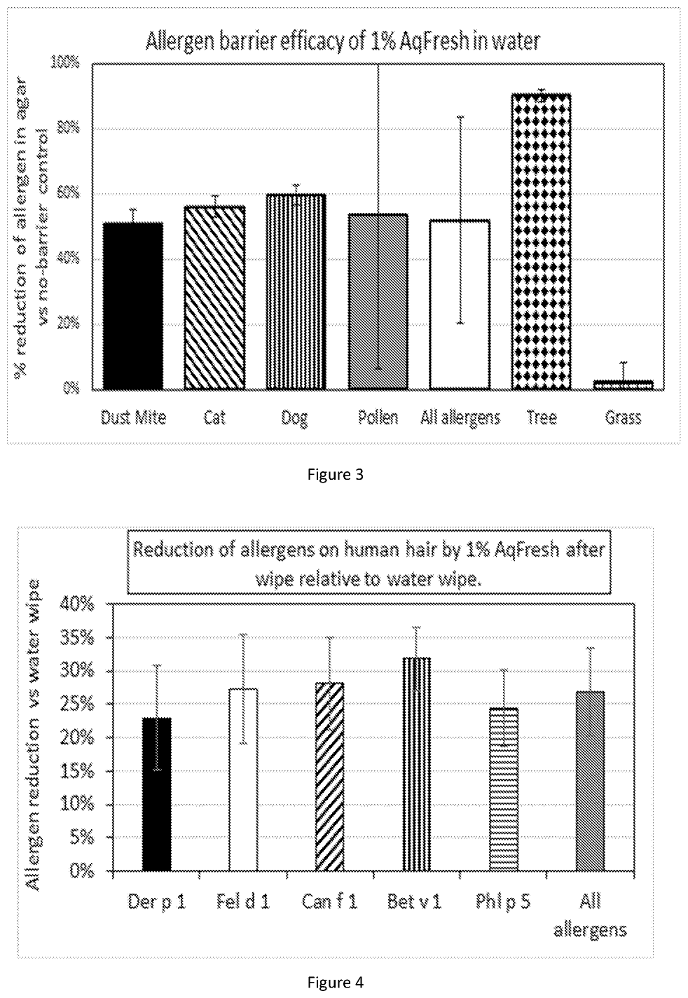

FIG. 3 which shows % w/w reduction allergen in agar versus no barrier control;

FIG. 4 which shows % w/w net reduction allergen on human hair after wiping with a cloth dampened with an aqueous suspension of 1% w/w AqFresh® compared to a cloth dampened with water; and

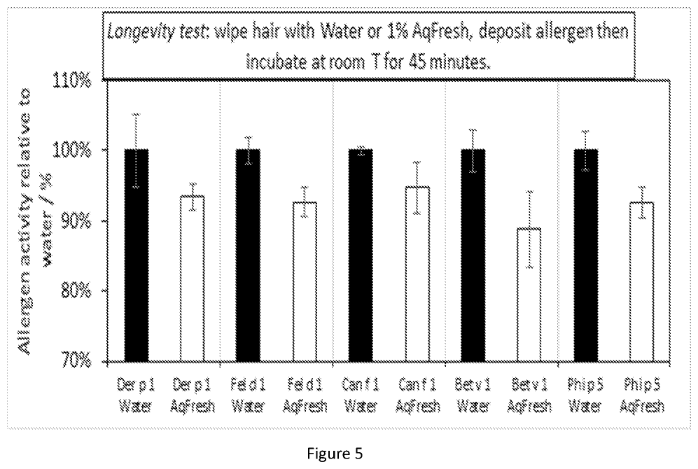

FIG. 5 which shows % w/w allergen on human hair which had previously been wiped with a cloth dampened with an aqueous suspension of 1% w/w AqFresh® compared to a water pre-treatment control.

### DETAILED DESCRIPTION OF THE INVENTION

In a first aspect of the invention, a non-therapeutic method for reducing or eliminating the allergic effect of an allergen is provided, the method comprising the step of bringing the allergen into physical contact with an effective amount of a cucurbituril, a derivative thereof, a variant thereof or a mixture thereof.

Cucurbituril is a member of the cavitand family and the general cucurbituril structure is based on the cyclic arrangement of glycoluril subunits linked by methylene bridges.

The preparation and purification of cucurbituril compounds is well described in the art. For example, Lagona et al. (Angewandte Chemie Int. Ed., 44, 31, 4844-4870 (2005)) review the synthesis and properties of cucurbituril compounds, including derivatives and analogues within the cucurbituril family.

For example, cucurbit[8]uril (CB[8]; CAS 259886-51-6) is a barrel shaped container molecule which has eight repeat glycoluril units and an internal cavity volume of 479 A³ (see structure below). CB[8] is readily synthesised using standard techniques and is available commercially (e.g. Sigma-Aldrich, MO USA).

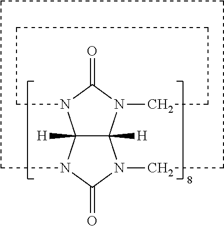

Cucurbituril is preferably selected from the group consisting of any one of cucurbit[4]uril to cucurbit[20]uril. Preferably the cucurbituril is provided as a mixture of different cucurbiturils. Thus cucurbituril may be present as a mixture of two or more cucurbiturils, wherein the mixture comprises at least two, preferably at least three different cucurbiturils selected from cucurbit[5]uril, cucurbit[6]uril, cucurbit[7]uril, and cucurbit[8]uril. Specifically, the mixture of cucurbiturils may comprise cucurbit[6]uril, cucurbit[7]uril and cucurbit[8]uril.

Other cucurbiturils may be present in addition to cucurbit[5]uril, cucurbit[6]uril, cucurbit[7]uril, and cucurbit[8]uril. For example, the mixture may further comprise cucurbiturils of CB[n] where n is an integer of 4 or 9 to 20, for example cucurbit[9]uril, cucurbit[10]uril, cucurbit[11]uril, etc. For example the mixture may further comprise cucurbit[4]uril or cucurbit[9]uril, or cucurbit[4]uril and cucurbit[9]uril.

When cucurbit[5]uril is present, the concentration of cucurbit[5]uril may be from about 0.01 to about 99% w/w, more particularly from about 0.05 to about 75% w/w, more particularly from about 0.05 to about 50% w/w, more particularly from about 0.05 to about 30% w/w, more particularly from about 0.05 to about 10% w/w, more particularly from about 0.05 to about 5% w/w, more particularly from about 0.05 to about 1% w/w, more particularly from about 0.05 to about 0.1% w/w based on the total weight of cucurbituril.

When cucurbit[6]uril is present, the concentration of cucurbit[6]uril may be from about 0.1 to about 99% w/w, more particularly from about 1 to about 75% w/w, more particularly from about 5 to about 65% w/w, more particularly from about 20 to about 65% w/w, more particularly from about 40 to about 65% w/w, more particularly from about 50 to about 60% w/w, based on the total weight of cucurbituril.

When cucurbit[7]uril is present, the concentration of cucurbit[7]uril may be from about 0.1 to 99% w/w, more particularly from about 5 to about 75% w/w, more particularly from about 10 to about 60% w/w, more particularly from about 10 to about 45% w/w, more particularly from about 15 to about 35% w/w, based on the total weight of cucurbituril. In one embodiment, the concentration of CB[7] is less than 45% w/w, based on the total weight of cucurbituril.

When cucurbit[8]uril is present, the concentration of cucurbit[8]uril may be from about 0.1 to 99% w/w, more particularly from about 0.5 to about 75% w/w, more particularly from about 1 to about 30% w/w, more particularly about 5 to about 25% w/w, more particularly from about 15 to about 25% w/w, based on the total weight of cucurbituril.

In one embodiment, the mixture of cucurbiturils comprises 35-75% w/w cucurbit[6]uril, 10-45% w/w cucurbit[7]uril and 5-30% w/w cucurbit[8]uril.

A variant of cucurbituril may include a structure having one or more repeat units that are structurally analogous to glycoluril. The repeat unit may include an ethylurea unit. Where all the units are ethylurea units, the variant is a hemicucurbituril. The variant may be a hemicucurbit[12]uril (shown below, see also Lagona et al. (Angewandte Chemie Int. Ed., 44, 31, 4844-4870 (2005))).

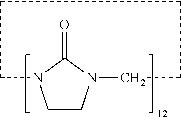

A derivative of a cucurbituril is a structure having one, two, three, four or more substituted glycoluril units. A substituted cucurbituril compound may be represented by the structure below:

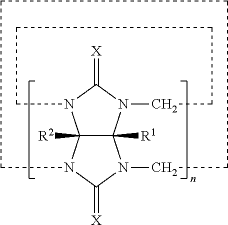

wherein:
n is an integer between 4 and 20;
and for each glycoluril unit:
each X is O, S or NR³, and
—R¹ and —R² are each independently selected from —H and the following optionally substituted groups: —R³, —OH, —OR³, —COOH, —COOR³, —NH₂, —NHR³ and —N(R³)₂ where —R³ is independently selected from C₁₋₂₀ alkyl, C₆₋₂₀ carboaryl, and C₅₋₂₀ heteroaryl, or where —R¹ and/or —R² is —N(R³)₂, both —R³ together form a C₅₋₇ heterocyclic ring; or together —R¹ and —R² are C₄₋₆ alkylene forming a C₆₋₈ carbocyclic ring together with the uracil frame.

In one embodiment, one of the glycoluril units is a substituted glycoluril unit. Thus, —R¹ and —R² are each independently —H for n−1 of the glycoluril units

In one embodiment, n is 5, 6, 7, 8, 9, 10, 11 or 12. In another embodiment, n is 5, 6, 7 or 8.

In one embodiment, each X is O. In another embodiment, each X is S.

In one embodiment, R¹ and R² are each independently H.

In one embodiment, for each unit one of R¹ and R² is H and the other is independently selected from —H and the following optionally substituted groups: —R³, —OH, —OR³, —COOH, —COOR³, —NH₂, —NHR³ and —N(R³)₂. In another embodiment, for one unit one of 8R¹ and R² is H and the other is independently selected from —H and the following optionally substituted groups: —R³, —OH, —OR³, —COOH, —COOR³, —NH₂, —NHR³ and —N(R³)₂. In this embodiment, the remaining glycoluril units are such that R¹ and R² are each independently H.

Preferably —R³ is C₁₋₂₀ alkyl, most preferably C₁₋₆ alkyl. The C₁₋₂₀ alkyl group may be linear and/or saturated. Each group —R³ may be independently unsubstituted or substituted. Preferred substituents are selected from: —R⁴, —OH, —OR⁴, —SH, —SR⁴, —COOH, —COOR₄, —NH₂, —NHR₄ and —N(R⁴)₂, wherein —R⁴ is selected from C₁₋₂₀alkyl, C₆₋₂₀carboaryl, and C₅₋₂₀ heteroaryl. The substituents may be independently selected from —COOH and —COOR₄.

In some embodiments, —R⁴ is not the same as —R³. In some embodiments, —R⁴ is preferably unsubstituted.

Where —R¹ and/or —R² is —OR³, —NHR³ or —N(R³)₂, then —R³ is preferably C₁₋₆ alkyl. In some embodiments, —R³ is substituted with a substituent —OR⁴, —NHR₄ or —N(R⁴)₂. Each —R⁴ is C₁₋₆ alkyl and is itself preferably substituted.

Derivatives or variants of cucurbiturils are often preferred because their solubility, dispersibility in a medium, and more generally their formulation and handling is improved over cucurbituril.

When the cucurbiturils, derivatives thereof or variants thereof come into contact with an allergen, it is thought that an allergen-cucurbituril complex forms which results in inactivating the ability of the allergen to provoke an allergic response.

Whilst cucurbituril, derivatives thereof and variants thereof exist as single molecular entities, particularly when in solution, they often, in practice, form particles, particularly porous particles. The particles can then form agglomerates (of particles). Whether in the form of single molecular entities, particles, or agglomerates of particles, it is expected that complexation between the cucurbituril, derivatives thereof and variants thereof and allergen will occur disrupting the ability of the allergen to provoke an allergic response.

In certain instances, the cucurbituril hosts a guest molecule, which is then released by molecular exchange when the cucurbituril complexes with an allergen. In one embodiment, the guest molecule is a molecule which is used in household care or personal care. In particular, the guest molecule is a fragrance molecule. The guest molecule is physically bound to the cucurbituril and is released when the cucurbituril complexes with an allergen. Suitable fragrance molecules are known to the skilled person in the art and described on pages 12-31 of WO 2017/141030 (Aqdot limited).

When forming a complex with a cucurbituril, the allergen is thought to be physically bound to the cucurbituril. The term ‘physically bound’ includes bonding by Van der Waals forces, hydrophobic bonding, and other types of physical bond such as hydrogen bonding. By being ‘physically bound’, complexes can be formed with a multitude of allergen having a multitude of chemical structures, even with allergen that has no reactive groups.

The cucurbituril-allergen complex may be a ternary or a binary complex. Thus, the cucurbituril may bind one or two allergen molecules via its cavity. Where a cucurbituril holds two allergen molecules, the allergen molecules may be the same or they may be different. A cucurbituril that is capable of binding two allergen molecules may also be capable of forming a stable binary complex with a single allergen molecule. The formation of a ternary guest-host complex is believed to proceed via an intermediate binary complex.

In one embodiment, the cucurbituril is capable of forming a ternary complex. For example, cucurbit[8]uril is capable of forming a ternary complex. In another embodiment, the cucurbituril is capable of forming a binary complex. cucurbit[8]uril may also form a binary complex.

In one embodiment, the cucurbituril is capable of forming ternary and binary complexes. For example, cucurbit[8]uril is capable of forming a ternary or a binary complex, depending upon the nature of the guest.

Preferably the effective amount of the cucurbituril, a derivative thereof, a variant thereof or a mixture thereof used in the non-therapeutic method of the first aspect of the invention, i.e. preferably in contact with the allergen, is in the range 0.00001-50, 0.00001-10, 0.00005-5, 0.00005-0.5, 0.00005-0.05, 0.00005-0.005% w/w.

Allergens originate from the Kingdom taxonomic classification Animalia, Fungi and Plantae. Selected volatile organic compounds (VOC's) such as terpenes or oxidised derivatives of terpenes are also considered allergens. Preferably the allergen originates from a source selected from the group consisting of pollen (from predominantly anemophilous plants (which rely on air dispersal of pollen) such as trees and grasses), an insect, a mould, a non-human mammal, a food, Hevea brasiliensis (Para rubber tree), a malodour and a fragrance.

The non-human mammal may be selected from the group consisting of cats, dogs, hamsters, guinea pigs, rabbits, chinchillas, horses, pigs, cows, sheep, goats, and rodents.

The pollen may be selected from the group consisting of birch pollen, sycamore pollen, chestnut pollen, alder pollen, hazel pollen, hickory pollen, oak pollen, timothy grass pollen, perennial rye grass, Orchard grass, Bermuda grass, and ragweed pollen.

The insect may be a house dust mite (for example (Dermatophagoides farinae (American house dust mite) and/or Dermatophagoides pteronyssinus (European house dust mite)) or a cockroach (for example the German cockroach).

The mould may be selected from the group consisting of the genus Alternaria (for example Alternaria alternata (Alternaria plant rot fungus)), Aspergillus (for example Aspergillus fumigatus (Common mould)), Cladosporium, and Penicillium (for example Penicillium chrysogenum).

The food may be selected from the group consisting of a fish, a crustacean, a cereal (for example oats, wheat, maize, rice, rye and barley), and peanut (Arachia hypogaea). Peanut protein allergens may be selected from the group consisting of Ara h 1, Ara h 2, Ara h 3, Ara h 5, Ara h 8 and Ara h 9.

The common protein allergens of Hevea brasiliensis (Para rubber tree) are Hey b 1, Hey b 3, Hey b 5, and Hey b 6.02.

The term “malodour” refers to unpleasant odours which are frequently encountered in everyday life and have a variety of origins. Typical malodours include odours that emanate from uncontrolled industrial activity, from human and pet body such as perspiration and excretion, from kitchen and food processing, from tobacco smoke, and from mould. Some of the most disturbing malodour for the human being are sweat; faecal; urine; wet pet; cooking odours, especially garlic, cabbage, fish and onion; and the like. Malodours may also emanate from the fatty acid and fatty acid derivatives present in consumer products, for example in soaps, detergents, shampoos, and conditioners. Other examples of particularly undesirable malodours are those produced by depilatory creams (sulphur compounds). WO 2017/141029 (Aqdot Limited) discloses examples of malodour molecules on pages 10-12.

WO 2017/141030 (Aqdot Limited) discloses examples of fragrances or fragrance molecules on pages 12 to 31.

Allergic reactions can also be caused by cross-reactivity where a compound from one source is similar to an allergen from another allergen source. As a result, the immune system of the host cannot distinguish between the compound and the allergen leading to an allergic reaction when the host is exposed to the compound. As such, suitable allergens include compounds which are similar to known allergens but which originate from different sources to that of the known allergens.

Specifically, the allergen may be selected from the group consisting of Der p 1 (from Dermatophagoides pteronyssinus (European house dust mite)), Der f 1 (from Dermatophagoides farinae (American house dust mite)), Der f 2, Der f 15, Der f 18, Fel d 1 (from Felis domesticus (F. catus) (Domestic cat)), Can f 1 (from Canis familiaris (C. lupus familiaris) (Domestic dog)), Mus m 1 (from Mus musculus (House mouse)), Rat n 1 (from Rattus norvegicus), Bla g 2 (from Blattella germanica (German cockroach)), Alt a 1 (from Alternaria alternata (Alternaria plant rot fungus)), Asp f 1 (from Aspergillus fumigatus (Common mould)), Bet v 1 (from Betula verrucosa (Betula pendula) (European white birch)), Phl p 5 (from Phleum pratense (Timothy grass)), and mixtures thereof. In particular, the allergen may be selected from the group consisting of Der p 1, Fel d 1, Can f 1, Bet v 1, and Phl p 5.

In one embodiment, the cucurbituril, a derivative thereof, a variant thereof or a mixture thereof is in the form of a particulate composition (for example in the form of a powder or as granules), a liquid or solid solution composition, a liquid or solid suspension composition (for example in the form of an emulsion), or a gel.

More specifically, the cucurbituril, a derivative thereof, a variant thereof or a mixture thereof may be in the form of a product selected from the group consisting of a home care product (for example in the form of a tablet, a wipe, a sponge, or a spray), a personal care product, a laundry care product (for example in the form of a tablet, a capsule or a spray), a paint or coating, personal protective equipment (such as gloves, a face mask, and a gown), and an air filter.

Thus the aforementioned composition or product may also include one or more additives known to those skilled in the art such as preservatives, dyes, pigments, sequestrants, surfactants, thickeners, rheology modifiers, polymers, and antioxidants. Methods of preparing the composition or product are well known to the person skilled in the art.

The product may be a cleansing composition (for example a laundry detergent, a hard surface cleaner, a soap, or a shampoo, a carpet cleaner), a liquid softener (for example a fabric or hair conditioner), a softener sheet, a fabric refresher, an air freshener, a deodorizing composition, a fine fragrance, a body mist, a cleansing wipe or mop, a styling gel, a humidity absorber, an air filter (non-woven and woven fabrics), a fabric finishing product, a brush, a pet product (for example a cleansing composition, a liquid softener, a brush, and a deodorizing composition), and the like.

In one embodiment, the composition or product may also provide malodour counteracting properties (i.e., a significant decrease of the intensity of the malodour perception by any person exposed to the source of the malodour) as described in WO 2017/141029 (Aqdot Limited).

Thus the composition or product may, in one embodiment, comprise one or more molecules, mixtures of molecules or polymers useful in counteracting malodour, in addition to cucurbituril. Suitable molecules, mixtures of molecules or polymers useful in counteracting malodour are known to the skilled person in the art and include other host-type molecules. For example, other members of the cavitand family such as cyclodextrins, calixarenes and crown ethers, can be used in combination with cucurbituril. Further suitable classes of molecules or compounds include charcoal.

In a second aspect of the invention, a cucurbituril, a derivative thereof, a variant thereof or a mixture thereof for use in treating and/or preventing the allergic effect of an allergen in a human or non-human animal (such as a mammal) is provided.

Specifically, treating the allergic effect of an allergen in a human or non-human animal can be achieved by complexation of histamine with a cucurbituril, a derivative thereof, a variant thereof or a mixture thereof. Histamine is part of an immune response to foreign pathogens and is involved in the inflammatory response and has a central role as a mediator of itching. Thus complexation of histamine with a cucurbituril, a derivative thereof, a variant thereof or a mixture thereof thereby to at least reduce the activity of histamine would be expected to reduce the effects of an allergic response.

Alternatively, a method for treating and/or preventing the allergic effect of an allergen in a human or non-human animal (such as a mammal) is provided, the method comprising administering to a subject in need an effective amount of a cucurbituril, a derivative thereof, a variant thereof or a mixture thereof.

The modes of administering a therapeutic agent to and determining the dosage regimen are well known to the skilled person in the art.

Alternatively, use of a cucurbituril, a derivative thereof, a variant thereof or a mixture thereof for the manufacture of a medicament for preventing the allergic effect of an allergen in a human or non-human animal (such as a mammal) is provided.

In one embodiment of the second aspect of the invention, the cucurbituril, a derivative thereof, a variant thereof or a mixture thereof may be in the form of a nasal or throat spray. Excipients typically used in a nasal spray include water, hydroxypropyl methylcellulose, succinic acid, disodium succinate, pyroglutamic acid (PCA), 2-phenylethanol, zinc EDTA, zinc acetate, and a nonionic surfactant or emulsifier such as Polysorbate 80.

The concentration of the cucurbituril, a derivative thereof, a variant thereof or a mixture thereof used in the second aspect of the invention, i.e., preferably in contact with the allergen, may be in the range 0.00001-50, 0.00001-10, 0.00005-5, 0.00005-0.5, 0.00005-0.05, 0.00005-0.005% w/w.

### Example 1: Animal Protein Allergens

Allergens were identified and quantified using the MARIA® allergen assay (Indoor Biotechnologies Inc., Virginia, USA) and a Luminex MAGPIX or xMAP 100/200 compact fluorescence detection system (Luminex Corporation, Texas, USA). Detailed instructions for use are provided with the assay.

The MARIA® allergen assay is a multiplex array for indoor allergens using the same (or equivalent) antibody combinations used in the conventional enzyme-linked immunosorbent assay (ELISA) used to quantify allergens. Capture antibodies are covalently coupled to polystyrene beads that are internally labelled with fluorophores. Combining different bead types with different antibodies allows simultaneous measurement of several allergens in a single test. Earle et al. (J. Allergy Clin. Immunol., 119, 2, 428 (2007)) provides a detailed description of the MARIA® allergen assay.

The Luminex MAGPIX or xMAP 100/200 compact fluorescence detection system analyses fluorescent bead-based assays by separating the beads using flow cytometry, exciting the beads using two lasers and analysing the output according to individual bead colour and colour intensity.

An allergen master mixture containing major dust mite allergen Der p 1, cat allergen Fel d 1, and dog allergen Can f 1 was prepared according to the assay instructions. Samples were prepared by mixing the allergen master mixture with the air freshener marketed as Oderase® (Aqdot Limited, Cambridge, England) comprising mixed cucurbiturils, or mixed cucurbiturils marketed as AgFresh® (Aqdot Limited, Cambridge, England) at a ratio of 1 part by weight allergen master mixture: 4 parts by weight Oderase® or AgFresh®, diluting to four different dilutions of neat, 1/10, 1/100 and 1/1000 and incubated for one hour on a roller mixer at room temperature (20-25 degrees centigrade). Oderase® comprises 0.5% w/w AgFresh®, water, xanthan gum and phenoxyethanol. AgFresh® comprises approximately 35-75% w/w cucurbit[6]uril, 10-45% w/w cucurbit[7]uril and 5-30% w/w cucurbit[8]uril and less than 1% w/w cucurbit[5]uril. The AgFresh® was provided as a 0.5% w/w aqueous dispersion.

Following incubation, the allergens in the samples and the master mixture were identified and quantified using a Luminex MAGPIX or xMAP 100/200 compact fluorescence detection system (Luminex Corporation, Texas, USA). Samples of Oderase® or AgFresh® with no allergen master mixture were also assessed to identify any background effects.

The allergen concentration at each dilution was compared to the concentration of the allergen master mixture with no cucurbituril to assess the amount of allergen remaining in each sample and calculate the percentage inhibition.

The assay results are summarised in Tables 1 to 6 below and in FIGS. 1 and 2.

TABLE 1
% inhibition of Dermatophagoides pteronyssinus (European house
dust mite) Der p 1 by mixed cucurbiturils of air freshener marketed as
Oderase^{RTM} at various dilution ratios (Neat product is 1 part by
weight allergen master mixture and 4 parts by weight Oderase^{RTM}).

| | Der p 1 | | | |
|---|---|---|---|---|
| | Average^{a} | | | |
| | concentration | SD | Inhibition | SD |
| | (ng/ml) | (ng/ml) | (%) | (%) |
| Allergen Master mix | 122.2 | 0.5 | 0 | 0.4 |
| Neat product | 88.7 | 4.5 | 27.5 | 3.7 |
| 1/10 dilution | 17.6 | 11.1 | 85.6 | 9.1 |
| 1/100 dilution | 4.4 | 7.6 | 96.4 | 6.2 |
| 1/1000 dilution | 7.5 | 13.0 | 93.9 | 10.6 |

^{a}Average of triplicate measurements.

With Der p 1, all the diluted Oderase® samples demonstrated a high level of inhibition although the neat sample did not inhibit allergen detection as well as the diluted samples. The highest level of allergen inhibition was with the 1/100 dilution, which led to a 96.4% reduction in detectable Der p 1allergen.

TABLE 2
% inhibition of Felis domesticus (F. catus) (Domestic cat)
Fel d 1 by mixed cucurbiturils of air freshener marketed as
Oderase^{RTM} at various dilution ratios (Neat product is 1 part by weight
allergen master mixture and 4 parts by weight Oderase^{RTM}).

| | Fel d 1 | | | |
|---|---|---|---|---|
| | Average^{a} | | | |
| | concentration | SD | Inhibition | SD |
| | (ng/ml) | (ng/ml) | (%) | (%) |
| Allergen Master mix | 163.6 | 4.5 | 0 | 2.7 |
| Neat product | 35.3 | 8.7 | 78.4 | 5.3 |
| 1/10 dilution | 47.1 | 0.4 | 71.2 | 0.3 |
| 1/100 dilution | 16.2 | 12.5 | 90.1 | 7.6 |
| 1/1000 dilution | 28.1 | 14.3 | 82.8 | 8.8 |

^{a}Average of triplicate measurements.

With Fel d 1, inhibition was quite similar across all the sample dilutions, with 1/100 demonstrating the greatest inhibition with a 90.1% reduction in detectable Fel d 1allergen.

TABLE 3
% inhibition of Canis familiaris (C. lupus familiaris) (Domestic dog)
Can f 1 by mixed cucurbiturils of air freshener marketed as Oderase^{RTM}
at various dilution ratios (Neat product is 1 part by weight allergen master
mixture and 4 parts by weight Oderase^{RTM}).

| | Can f 1 | | | |
|---|---|---|---|---|
| | Average^{a} | | | |
| | concentration | SD | Inhibition | SD |
| ODERASE | (ng/ml) | (ng/ml) | (%) | (%) |
| Allergen Master mix | 92.5 | 0.9 | 0 | 1.0 |
| Neat product | 49.1 | 14.2 | 46.9 | 15.3 |
| 1/10 dilution | 10.9 | 0.5 | 88.2 | 0.5 |
| 1/100 dilution | 2.3 | 1.1 | 97.5 | 1.2 |
| 1/1000 dilution | 1.5 | 1.2 | 98.3 | 1.3 |

^{a}Average of triplicate measurements.

With Can f 1, a very similar pattern of inhibition to Der p 1 was seen with the neat sample not demonstrating quite as good inhibition as the diluted product. The 1/100 and 1/1000 dilutions demonstrated a 97.5% and a 98.3% reduction in Can f 1 allergen detection respectively.

TABLE 4
% inhibition of Dermatophagoides pteronyssinus (European
house dust mite) Der p 1 by mixed cucurbiturils of
AqFresh^{RTM} at various dilution ratios (Neat product is 1 part
by weight allergen master mixture and 4 parts by weight
AqFresh^{RTM} provided as a 0.5% w/w aqueous solution).

| | Der p 1 | | | |
|---|---|---|---|---|
| | Average^{a} | | | |
| | concentration | SD | Inhibition | SD |
| | (ng/ml) | (ng/ml) | (%) | (%) |
| Allergen Master mix | 122.2 | 0.5 | 0 | 0.4 |
| Neat product | 26.1 | 3.9 | 78.6 | 3.2 |
| 1/10 dilution | 2.5 | 0.8 | 97.9 | 0.7 |
| 1/100 dilution | 0 (*<LOD) | 0.0 | 100 | 0.0 |
| 1/1000 dilution | 56.2 | 4.0 | 54 | 3.3 |

^{a}Average of triplicate measurements.
*LOD is limit of detection

With Der p 1, all dilutions of AqFresh® resulted in allergen detection being inhibited. At the 1/100 dilution, no allergen could be detected in the samples (the result was below the limit of detection of the assay) and therefore was deemed to inhibit allergen detection by 100%. The least inhibition was seen at 1/1000 dilution.

TABLE 5
% inhibition of Felis domesticus (F. catus) (Domestic cat) Fel
d 1 by mixed cucurbiturils of AqFresh^{RTM} at various dilution ratios
(Neat product is 1 part by weight allergen master mixture and 4 parts by
weight AqFresh^{RTM} provided as a 0.5% w/w aqueous solution).

| | Fel d 1 | | | |
|---|---|---|---|---|
| | Average^{a} | | | |
| | concentration | SD | Inhibition | SD |
| | (ng/ml) | (ng/ml) | (%) | (%) |
| Allergen Master mix | 163.6 | 4.5 | 0 | 2.7 |
| Neat product | 110.2 | 7.6 | 32.6 | 4.6 |
| 1/10 dilution | 67.3 | 12.2 | 58.9 | 7.5 |
| 1/100 dilution | 4.4 | 5.6 | 97.3 | 3.4 |
| 1/1000 dilution | 20.7 | 0.1 | 87.4 | 0.1 |

^{a}Average of triplicate measurements.

Inhibition of Fel d 1 was seen across all dilutions of AgFresh®, however this was not quite as consistent as with Oderase® 1/100 again demonstrated the greatest inhibition with a 97.3% reduction in detectable Fel d 1 allergen.

TABLE 6
% inhibition of Canis familiaris (C. lupus familiaris)
(Domestic dog) Can f 1 by mixed cucurbiturils of AqFresh^{RTM}
at various dilution ratios (Neat product is 1 part by weight allergen
master mixture and 4 parts by weight AqFresh^{RTM} provided
as a 0.5% w/w aqueous solution).

| | Can f 1 | | | |
|---|---|---|---|---|
| | Average^{a} | | | |
| | concentration | SD | Inhibition | SD |
| | (ng/ml) | (ng/ml) | (%) | (%) |
| Allergen Master mix | 92.5 | 0.9 | 0 | 1.0 |
| Neat product | 68 | 2.7 | 26.4 | 2.9 |
| 1/10 dilution | 34.1 | 6.6 | 63.2 | 7.1 |
| 1/100 dilution | 0.3 | 0.2 | 99.8 | 0.2 |
| 1/1000 dilution | 13.9 | 1.4 | 85 | 1.5 |

^{a}Average of triplicate measurements.

Inhibition of Can f 1 by AqFresh® was not as comprehensive across all dilutions as Oderase® however at 1/100 dilution, the greatest inhibition was observed with a percentage reduction in detectable Can f 1 allergen of 99.8%.

No fluorescence and hence no matrix affects were observed when Oderase® or AqFresh® alone was analysed.

### Example 2: Pollen Protein Allergens

The assay described in Example 1 was repeated with European white birch pollen allergen Bet v 1 and Timothy grass (Phleum pratense) pollen allergen Phl p 5 and the results are summarised in Tables 7 to 9 below.

TABLE 7
% inhibition of Betula verrucosa (Betula pendula) (European
white birch) Bet v 1 by mixed cucurbiturils of air freshener marketed as
Oderase^{RTM} at various dilution ratios (Neat product is 1 part by
weight allergen master mixture and 4 parts by weight Oderase^{RTM}).

| | Bet v 1 | | | |
|---|---|---|---|---|
| | Average^{a} | | | |
| | concentration | SD | Inhibition | SD |
| | (ng/ml) | (ng/ml) | (%) | (%) |
| Allergen Master mix | 184.4 | 0.5 | 0.0 | 0.30 |
| Neat product | 125.5 | 4.4 | 32.0 | 2.40 |
| 1/10 dilution | 24.1 | 0.1 | 87.0 | 0.10 |
| 1/100 dilution | 30.7 | 0.4 | 83.4 | 0.20 |

^{a}Average of duplicate measurements.

Inhibition of Bet v 1 by Oderase® was demonstrated at all dilutions of Oderase® However the neat product did not inhibit allergen detection as well as the diluted products. Both diluted products demonstrated a similarly high level of inhibition.

TABLE 8
% inhibition of Phleum pratense (Timothy grass) Phl p 5 by
mixed cucurbiturils of air freshener marketed as Oderase^{RTM} at
various dilution ratios (Neat product is 1 part by weight allergen
master mixture and 4 parts by weight Oderase^{RTM}).

| | Phl p 5 | | | |
|---|---|---|---|---|
| | Average^{a} | | | |
| | concentration | SD | Inhibition | SD |
| | (ng/ml) | (ng/ml) | (%) | (%) |
| Allergen Master mix | 212.1 | 1.5 | 0.0 | 0.7 |
| Neat product | 191.5 | 12.8 | 9.7 | 6.0 |
| 1/10 dilution | 69.1 | 8.0 | 67.4 | 3.8 |
| 1/100 dilution | 14.6 | 1.1 | 93.1 | 2.5 |

^{a}Average of duplicate measurements.

Inhibition of Phl p 5 by Oderase® was demonstrated at all dilutions of Oderase® although only at a low level by the neat product. However as the dilution of the Oderase® increased, so did the percentage inhibition, with 1/100 demonstrating inhibition of over 90%.

TABLE 9
% inhibition of Betula verrucosa (Betula pendula) (European
white birch) Bet v 1 by mixed cucurbiturils of AqFresh^{RTM} at
various dilution ratios (Neat product is 1 part by weight allergen
master mixture and 4 parts by weight AqFresh^{RTM} provided
as a 0.5% w/w aqueous solution).

| | Bet v 1 | | | |
|---|---|---|---|---|
| | Average^{a} | | | |
| | concentration | SD | Inhibition | SD |
| | (ng/ml) | (ng/ml) | (%) | (%) |
| Allergen Master mix | 184.4 | 1.07 | 0 | 0.60 |
| Neat product | 100.1 | 0.1 | 45.05 | 0.10 |
| 1/10 dilution | 49.09 | 7.5 | 73.05 | 4.10 |
| 1/100 dilution | 6.45 | 2.3 | 96.46 | 1.20 |

^{a}Average of duplicate measurements.

Inhibition of Bet v 1 by AqFresh® was demonstrated at all dilutions. As the dilution of the product increased, so did the percentage inhibition, with the 1/100 dilution demonstrating inhibition of over 90%.

### Example 3: The Effect of a 1% w/w Aqueous Suspension of AgFresh® in Preventing Diffusion of Cat (Fel d 1), Dog (can f 1), House Dust Mite (Der p 1), Tree (Bet v 1) and Grass (Phl p 5) Allergens in a Skin Model

To replicate a skin surface, small (about 1×1 cm) rectangles of agar (1.5% with 0.9% saline solution) were cut and transferred to microscope slides. Two lines of Vaseline® were drawn to prevent diffusion of allergens to the agar around the AqFresh® suspension. The suspension was applied to each slide adjacent to the moist agar acting as the ‘barrier’. Additionally, a control sample containing no barrier was included. The suspension samples were prepared in quadruple and the control sample were prepared in triplicate. A cover slip was added on top of the slide and a liquid allergen master mix as previously described was pipetted under the cover slip, adjacent to the barrier. The allergen master mix contained purified forms of cat (Fel d 1), dog (Can f 1), house dust mite (Der p 1), tree (Bet v 1) and grass (Phl p 5) allergens.

The slides were incubated in a humidified chamber for two hours at room temperature (about 20-25 degrees centigrade) before the agar was carefully removed from the slides and transferred to microcentrifuge tubes containing 0.5 ml of phosphate buffer solution-Tween. Each sample of agar was extracted by brief vortex (30 seconds) followed by gentle shaking overnight at room temperature and the amount of allergen within each extracted sample was measured using Indoor Biotechnologies Inc. (Virginia, USA) MARIA® assay previously described.

The results are illustrated in FIG. 3 which shows % w/w reduction of each of cat (Fel d 1), dog (Can f 1), house dust mite (Der p 1), tree (Bet v 1), grass (Phl p 5), the result of the sum of the tree (Bet v 1) and the grass (Phl p 5) pollens, and the sum of all allergens in agar versus no barrier control.

### Example 4: The Effect of a 1% w/w Aqueous Suspension of AgFresh® in Reducing Levels of Cat (Fel d 1), Dog (can f 1), House Dust Mite (Der p 1), Tree (Bet v 1) and Grass (Phl p 5) Allergens on Cat/Pet Fur/Hair

The hair used was 8 inch long ‘Julia Virgin 4 Bundles Straight Peruvian Hair Weave Deals Human Hair Extensions’ (https://www.juliahair.com/julia-virgin-4-bundles-straight-peruvian-hair-weave-deals-human-hair-extensions.html). The allergen master mixture was prepared as previously described and contained the following allergens: cat (Fel d 1), dog (Can f 1), dust mite (Der p 1), grass pollen (Phl p 5) and tree pollen (Bet v 1).

All hair and cotton cloths (7×15 cm) were washed in warm water (no detergent) prior to use to remove any potential allergen contamination. Each test sample of hair comprised three layers of hair (7 cm wide) clipped together to add more ‘depth’ and more hair surface for the allergens to attach.

600 μl of the allergen master mixture was added evenly across the hair using a multichannel pipette. 10 ml of water or the 1% w/w aqueous suspension of AgFresh® was used to dampen the cotton cloths which were then used to wipe the hair. All hair samples were extracted in 20 ml of PBS+0.05% Tween at room temperature (20-25 degrees centigrade) in ziplock bags placed flat. The samples were extracted for a total of 2 hours and were massaged every 20 minutes to maximise the extraction efficiency.

In order to determine the effectiveness of allergen removal from hair using a 1% w/w aqueous suspension of AgFresh®:
1. The allergen master mixture was applied onto the hair as described above and left to dry for 45 minutes.
2. A cotton cloth was dampened with 10 ml of water (control) or 1% w/w AqFresh in water and used to wipe the hair from its fixture point outwards until all areas/fibres were damp but not wet. All hair samples were wiped 15 times following the same pattern.
3. The hair and the cloths were left on the benchtop for 30 minutes.
4. To determine the allergen concentration remaining on the hair, all samples were extracted using a modified Indoor Biotechnlogies Inc (Virginia, USA) assay for allergen extraction and analysed using the MARIA® allergen assay previously described.
5. All controls and samples were tested in triplicate.

In order to determine the on-going reduction of allergens using a 1% w/w aqueous suspension of AgFresh®:
1. A cotton cloth was dampened with 10 ml of water (control) or 1% w/w AgFresh® in water and used to lightly wipe each hair sample from its fixture point outwards until all areas/fibres were damp but not wet. All hair samples were wiped 15 times following the same pattern.
2. The allergen master mixture was applied to the damp hair samples (as described before) and allowed to incubate at room temperature (20-25 degrees centigrade) for 45 minutes.
3. To determine the allergen concentration, the hair samples were extracted using a modified Indoor Biotechnlogies Inc (Virginia, USA) assay for allergen extraction and analysed using the MARIA® allergen assay previously described.
4. All controls and samples were tested in triplicate.

The results are illustrated in FIGS. 4 and 5 which show respectively the percentage w/w net reduction in the level of each allergen after wiping with a cloth comprising 1% w/w AqFresh® in water compared with just water as a control and the percentage w/w allergen for each allergen when a hair sample pre-treated with 1% w/w AqFresh® in water compared to a water pre-treatment control.

Compared to water alone addition of 1% w/w AqFresh® in water improved immediate removal of all allergens from 23.5% (Der p 1) to 32.3% (Bet v 1), where the difference in levels of Bet v 1 was statistically significant using unpaired t-test (p<0.05). Compared to water alone, the reduction in allergen weight for each allergen on pre-treatment of the hair samples with 1% w/w AgFresh® in water was about 8%.

1. A non-therapeutic method for reducing or eliminating the allergic effect of an allergen comprising the step of bringing the allergen into physical contact with an effective amount of a cucurbituril, a derivative thereof, a variant thereof or a mixture thereof.

2. A non-therapeutic method for reducing or eliminating the allergic effect of an allergen according to claim 1, wherein the cucurbituril is selected from the group consisting of any one of cucurbit[4]uril to cucurbit[20]uril.

3. A non-therapeutic method for reducing or eliminating the allergic effect of an allergen according to claim 1, comprising the step of bringing the allergen into physical contact with a mixture of cucurbiturils.

4. A non-therapeutic method for reducing or eliminating the allergic effect of an allergen according to claim 3, wherein the mixture of cucurbiturils comprises cucurbit[6]uril, cucurbit[7]uril and cucurbit[8]uril.

5. A non-therapeutic method for reducing or eliminating the allergic effect of an allergen according to claim 4, wherein the mixture of cucurbiturils comprises 35-75% w/w cucurbit[6]uril, 10-45% w/w cucurbit[7]uril and 5-30% w/w cucurbit[8]uril.

6. A non-therapeutic method for reducing or eliminating the allergic effect of an allergen according to claim 1, wherein the allergen originates from a source selected from the group consisting of pollen, an insect, a mould, a non-human mammal, a food, Hevea brasiliensis, a malodour, and a fragrance.

7. A non-therapeutic method for reducing or eliminating the allergic effect of an allergen according to claim 6, wherein the non-human mammal is selected from the group consisting of cats, dogs, hamsters, guinea pigs, rabbits, chinchillas, horses, pigs, cows, sheep, goats, and rodents.

8. A non-therapeutic method for reducing or eliminating the allergic effect of an allergen according to claim 6, wherein the pollen is selected from the group consisting of birch pollen, sycamore pollen, chestnut pollen, alder pollen, hazel pollen, hickory pollen, oak pollen, timothy grass pollen, perennial rye grass, orchard grass, Bermuda grass, and ragweed pollen.

9. A non-therapeutic method for reducing or eliminating the allergic effect of an allergen according to claim 6, wherein the insect is a house dust mite or a cockroach.

10. A non-therapeutic method for reducing or eliminating the allergic effect of an allergen according to claim 6, wherein the mould is selected from the group consisting of the genus Alternaria, Aspergillus, Cladosporium and Penicillium.

11. A non-therapeutic method for reducing or eliminating the allergic effect of an allergen according to claim 1, wherein the allergen is selected from the group consisting of Der p 1, Der f 1, Der f 2, Der f 15, Der f 18, Feld 1, Can f 1, Mus m 1, Rat n 1, Bla g 2, Alta 1, Asp f 1, Bet v 1, Phl p 5, and mixtures thereof.

12. A non-therapeutic method for reducing or eliminating the allergic effect of an allergen according to claim 11, wherein the allergen is selected from the group consisting of Der p 1, Fel d 1, Can f 1, Bet v 1, and Phl p 5.

13. A non-therapeutic method for reducing or eliminating the allergic effect of an allergen according to claim 1, wherein the cucurbituril, a derivative thereof, a variant thereof or a mixture thereof is in the form of a particulate composition, a liquid or solid solution composition, or a liquid or solid suspension composition.

14. A non-therapeutic method for reducing or eliminating the allergic effect of an allergen according to claim 1, wherein the cucurbituril, a derivative thereof, a variant thereof or a mixture thereof is in the form of a product selected from the group consisting of a home care product, a personal care product, a laundry care product, a paint or coating, and an air filter.

15. A cucurbituril, a derivative thereof, a variant thereof or a mixture thereof for use in treating and/or preventing the allergic effect of an allergen in a human or non-human animal.
